(19) United States
(12) Patent Application Publication
(10) Pub. No.: US 2024/0353339 A1
(43) Pub. Date: Oct. 24, 2024

(54) DEVELOPMENT OF FLEXIBLE PLASMONIC MEMBRANE-BASED SERS PLATFORM FOR MONITORING THE HEALING

(71) Applicant: Agency for Science, Technology and Research, Singapore (SG)

(72) Inventors: Malini Olivo, Singapore (SG); Jayakumar Perumal, Singapore (SG); Dinish Unnimadhava Kurup Soudamini Amma, Singapore (SG)

(21) Appl. No.: 18/683,345

(22) PCT Filed: Aug. 19, 2022
(86) PCT No.: PCT/SG2022/050590

§ 371 Date: Feb. 13, 2024

(30) Foreign Application Priority Data
Aug. 20, 2021 (SG) 10202109134Q

(51) Int. Cl. G01N 21/65; G01N 33/543; G01N 33/68

(52) U.S. Cl. CPC G01N 21/658; G01N 33/54346; G01N 33/54373; G01N 33/6893; G01N 2333/525; G01N 2333/54; G01N 2333/96419

(57) ABSTRACT

A biocompatible SERS-active polymer membrane configured to detect biomarkers in a sample. The biocompatible SERS-active polymer membrane includes a flexible and porous polymer membrane and SERS-active nanoparticles formed on the flexible and porous polymer membrane, where the flexible and porous polymer membrane includes cellulose or an elastomeric polymer. Also disclosed herein is a method of forming the biocompatible SERS-active polymer membrane and a method of determining a state of wound healing in a diabetic individual involving the use of the biocompatible SERS-active polymer membrane.

### CROSS-REFERENCE TO RELATED APPLICATIONS

This application claims the benefit of priority of Singapore patent application Ser. No. 10/202,109134Q, filed 20 Aug. 2021, the content of it being hereby incorporated by reference in its entirety for all purposes.

### TECHNICAL FIELD

The present disclosure relates to a biocompatible SERS-active polymer membrane configured to detect biomarkers and its uses in detecting biomarkers associated with wound healing. The present disclosure also relates to a method of forming the biocompatible SERS-active polymer membrane.

### BACKGROUND

Wound healing in the skin may include several complicated time-dependant biological processes, such as haemostasis, inflammation, proliferation, repair, and remodelling, and these may be influenced by environmental factors. Following an injury, the proteolytic cascade may initiate a process of blood clotting, which in turn activates platelet degranulation that releases a surge of pre-formed growth factors and cytokines stored in the alpha granules. The growth factors and cytokines may diffuse rapidly from the wound into surrounding tissues and chemotactically draw inflammatory cells into the wounded area. The neutrophils and macrophages may engulf and break down bacteria and release proteases such as neutrophil elastase and matrix metalloproteinase eight (MMP-8). These proteases may play a role in introducing wound healing by proteolytically removing impaired extracellular matrix components for new extracellular matrix molecules in order for wound healing to progress desirably. Although matrix metalloproteinase levels may decrease over the period of normal wound healing, chronic wounds tend to have a considerably higher level of proteases and pro-inflammatory cytokines, such as tumour necrosis factor alpha (TNF-α), interleukin one (IL1), along with lower levels of growth factors.

However, there appears to be limited diagnostic means to assess the status in wound healing (e.g. diabetes mellitus (DM) wound healing) and to identify complications that may impair healing. The ability to evaluate critical biological activities or impairments may help guide clinicians in predicting wound outcomes, and in turn helps in selecting the most suitable and cost-effective treatment plans.

For efficacious treatment of DM wounds, diagnostic tests that have been developed traditionally assesses biological factors, such as biomarkers, to aid clinicians in formulating the most appropriate treatment plans. Biomarkers, such as TNF-α, MMP-8, MMP-9 and interleukins, may be measured objectively and assessed as an indicator of standard biological characteristic that may be used to evaluate the progress of wound healing or effectiveness of the treatment plans. Traditionally, several methods were used to evaluate the levels of biomarkers, such as enzyme-linked immunosorbent assays (ELISAs) and liquid chromatography mass spectrometry (LC-MS). Traditional ELISAs may have been commonly used for clinical biomarkers determination with detection limits nearing 1 pg/ml for some protein biomarkers, but this method tends to be laboriously time and effort consuming, costly, and not sufficiently sensitive for point-of-care detection of multiple biomarkers. LC-MS, on the other hand, may be able to carry out multiple biomarkers measurements with acceptable sensitivity but tends to be time consuming and tends to involve expensive equipment that may be operably further time consuming and complex for point of care detection.

There is thus a need to provide for a solution that addresses one or more of the limitations mentioned above.

### SUMMARY

In a first aspect, there is provided for a biocompatible SERS-active polymer membrane configured to detect biomarkers in a sample, comprising:
a flexible and porous polymer membrane; and
SERS-active nanoparticles formed on the flexible and porous polymer membrane, wherein the flexible and porous polymer membrane comprises cellulose or PDMS.

In another aspect, there is provided a method of forming the biocompatible SERS-active polymer membrane described in various embodiments of the first aspect, the method comprising:
providing a flexible and porous polymer membrane, wherein the flexible and porous polymer membrane comprises cellulose or an elastomeric polymer; and
depositing SERS-active nanoparticles on the flexible and porous polymer membrane.

Various embodiments relate to the biocompatible SERS-active polymer membrane described in various embodiments of the first aspect for use in determining a state of wound healing in a diabetic individual. The SERS-active nanoparticles, which may comprise silver nanoparticles as one non-limiting example, may be incorporated on the flexible and porous polymer membrane. Advantageously, such SERS-active silver nanoparticles not only provide for Raman signal enhancement but also confer an anti-bacterial effect, hence further providing a therapeutic benefit.

In another aspect, there is provided a method of determining a state of wound healing in a diabetic individual, the method comprising:
contacting a sample extracted from a wound of the diabetic individual with a thiolating agent to have one or more biomarkers suspected to be contained in the sample form one or more modified proteins;
incubating the biocompatible SERS-active polymer membrane described in various embodiments of the first aspect with the one or more modified proteins to immobilise the one or more modified proteins on SERS-active nanoparticles of the biocompatible SERS-active polymer membrane;
providing one or more Raman probes attached with one or more antibodies, wherein each of the one or more antibodies binds specifically to one corresponding modified protein from the one or more modified proteins;
contacting the one or more Raman probes with the biocompatible SERS-active polymer membrane having the one or more modified proteins immobilised thereon; and
subjecting the biocompatible SERS-active polymer membrane to SERS spectroscopy to generate one or more SERS signals corresponding to the one or more biomarkers, respectively.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1A is a general schematic illustration showing cellulose fibers (CF) SERS substrate fabrication and treatment of the cellulose fibers SERS substrate for biomarker proteins sensing.

FIG. 1B is a schematic illustration showing cellulose fibers SERS substrate fabrication.

FIG. 1C is a schematic illustration showing treatment of cellulose fibers SERS substrate for biomarker proteins sensing.

FIG. 2A shows a representative SERS spectrum of 4-aminothiophenol (4-ATP).

FIG. 2B shows a representative SERS spectrum of 2-naphthalenethiol (2-NT).

FIG. 3A shows a FE-SEM image of the CF SERS substrate fabricated by Ag deposition at 10 mA with a deposition time of 30 s. Scale bar (in white) denotes 200 nm. Scale bar (in black) denotes 100 nm.

FIG. 3B shows a FE-SEM image of the CF SERS substrate fabricated by Ag deposition at 10 mA with a deposition time of 40 s. Scale bar (in white) denotes 200 nm. Scale bar (in black) denotes 100 nm.

FIG. 3C shows a FE-SEM image of the CF SERS substrate fabricated by Ag deposition at 10 mA with a deposition time of 50 s. Scale bar (in white) denotes 200 nm. Scale bar (in black) denotes 100 nm.

FIG. 3D shows a FE-SEM image of the CF SERS substrate fabricated by Ag deposition at 10 mA with a deposition time of 60 s. Scale bar (in white) denotes 200 nm. Scale bar (in black) denotes 100 nm.

FIG. 3E shows a FE-SEM image of the CF SERS substrate fabricated by Ag deposition at 10 mA with a deposition time of 70 s. Scale bar (in white) denotes 200 nm. Scale bar (in black) denotes 100 nm.

FIG. 3F shows a FE-SEM image of the CF SERS substrate fabricated by Ag deposition at 10 mA with a deposition time of 80 s. Scale bar (in black) denotes 100 nm.

FIG. 4A shows a SEM image of the surface morphology of Ag-coated cellulose fibers substrates at 20 mA with a deposition time of 20 s. Scale bar (in black) denotes 100 nm.

FIG. 4B shows a SEM image of the surface morphology of Ag-coated cellulose fibers substrates at 20 mA with a deposition time of 30 s. Scale bar (in black) denotes 100 nm.

FIG. 4C shows a SEM image of the surface morphology of Ag-coated cellulose fibers substrates at 20 mA with a deposition time of 40 s. Scale bar (in black) denotes 100 nm.

FIG. 4D shows a SEM image of the surface morphology of Ag-coated cellulose fibers substrates at 20 mA with a deposition time of 50 s. Scale bar (in black) denotes 100 nm.

FIG. 4E shows a SEM image of the surface morphology of Ag-coated cellulose fibers substrates at 20 mA with a deposition time of 60 s. Scale bar (in black) denotes 100 nm.

FIG. 4F shows a SEM image of the surface morphology of Ag-coated cellulose fibers substrates at 20 mA with a deposition time of 80 s. Scale bar (in black) denotes 100 nm.

FIG. 5A is a plot showing the maximum SERS signal enhancement obtained for the cellulose fibers SERS substrate with approximately 60 nm Ag film thickness.

FIG. 5B is a plot that shows the uniformity of the substrate signal enhancement across the substrate.

FIG. 6 shows evaluation of ideal Ag deposition condition at 10 mA for the CF SERS substrate. This plot also shows the maximum SERS signal enhancement that was obtained for the cellulose fibers SERS substrate.

FIG. 7A shows SERS mapping of 2-NT on an immobilised flexible CF membrane. FIG. 7A shows the optical view of the CF membrane. Scale bar denotes 200 μm.

FIG. 7B shows SERS mapping of 2-NT on an immobilised flexible CF membrane. FIG. 7B shows the mapping view overlaid on the optical view of FIG. 7A. The bright red colour (marked by the rectangle) denotes the SERS intensity of representative Raman peak at 1066 cm⁻¹. SERS mapping shows minimum variation in SERS enhancement for 2-NT on the Ag SNP substrate. Scale bar denotes 200 μm.

FIG. 8A shows calibration plot of MMP-9 (black circle data points). Square data points indicate the detection of spiked concentrations of MMP-9 from the simulated serum samples using respective calibration plots.

FIG. 8B shows calibration plot of TNF-α biomarkers (black circle data points). Square data points indicate the detection of spiked concentrations of TNF-α biomarkers from the simulated serum samples using respective calibration plots.

FIG. 9A shows a calibration plot of IL-1α biomarkers.

FIG. 9B shows a calibration plot of IL-1β biomarkers.

FIG. 10 is a table showing the recovery studies of MMP-9 and TNF-α in spiked samples.

FIG. 11A shows SEM images of polydimethylsiloxane (PDMS) stretched and plain (unstretched) at 100×, 150× and 300× magnification.

FIG. 11B shows the optical image of plain PDMS membrane to the left and the final plasmonic silver deposited PDMS on the right.

FIG. 12 shows calibration plots for different biomarkers using PDMS membrane SERS substrate.

FIG. 13 is a schematic of an experimental workflow for a biosensing protocol of the present disclosure.

FIG. 14 is a table comparing the advantages and applications of traditional ELISA method and the present SERS-based method.

FIG. 15 shows a flexible biosensor of the present disclosure configured as a band aid with multiplexing capability to detect more than one biomarker at a given time.

FIG. 16 is a schematic illustration for SERS Multiplex biosensing platform for multiple wound biomarker sensing.

### DETAILED DESCRIPTION

The following detailed description refers to the accompanying drawings that show, by way of illustration, specific details and embodiments in which the present disclosure may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a biocompatible SERS-active polymer membrane configured to detect biomarkers in a sample. The biocompatible SERS-active polymer membrane can comprise a flexible and porous polymer membrane having SERS-active nanoparticles formed thereon. In various embodiments, the flexible and porous polymer membrane may comprise cellulose or an elastomeric polymer. The present disclosure also relates to a method of forming the biocompatible SERS-active polymer membrane and use of the biocompatible SERS-active polymer membrane in determining a state of wound healing in a subject, e.g. a diabetic individual.

The biocompatible SERS-active polymer membrane is advantageous for determining a state of wound healing in a subject. To determine the state of wound healing in the subject, the biocompatible SERS-active polymer membrane is compatibly used in combination with surface enhanced Raman spectroscopy (SERS), wherein the flexible and porous polymer membrane can comprise a polydimethylsiloxane (PDMS) or cellulose fiber (CF) based membrane as non-limiting examples, and configured to be used as a viable point of care platform to monitor the changes of various cytokines, proteases and other small molecules as biomarkers that serve as an indicator on the state of the wound and the progress of the wound healing.

In the present disclosure, the inclusion of SERS is demonstrated as a sensitive and selective method to detect various wound biomarkers because of its ability to produce fingerprint spectra of its analyte rapidly at high resolution. Various flexible and transparent membrane-based SERS substrate (e.g. PDMS and cellulose) decorated with randomly arranged metal (e.g. silver or gold) nanostructures are demonstrated in the examples section of the present disclosure.

Advantageously, the biocompatible SERS-active polymer membrane, such as the PDMS and cellulose-based SERS membranes, can be used for sample collection in wet condition of wound exudates and by employing a robust bio-sensing method to detect the biomarkers. The terms “membrane” and “substrate” are used interchangeably in the present disclosure.

In the case of small molecules, the biocompatible SERS-active polymer membrane provides for a label-free direct detection approach that can be used without further sample processing followed by data processing to unmix individual biomarker data by means of machine learning.

The biocompatible SERS-active polymer membrane is advantageous for wound management. Wound management is stretching the limits of health systems globally, challenging clinicians to evaluate the effectiveness of their treatments and deliver appropriate care to their patients. Visual inspection and manual measurement of wound size is subjective, often inaccurate and inconsistent. Non-healing wounds constitute a major portion of the healthcare spending in developed nations and it also overloads the healthcare workers. A non-healing wound is deemed as a wound that does not heal within five to eight weeks, even after following a prescribed treatment and/or wound management regime to take care of it.

Advantageously, the biocompatible SERS-active polymer membrane is a flexible SERS substrate that can be configured as and/or form part of wound dressing. Said differently, the present disclosure provides for the use of the SERS-active polymer membrane described in various embodiments of the first aspect in the manufacture of a wound dressing. The wound dressing can be used to determine the state of wound healing in a subject, e.g. a diabetic individual.

The biocompatible SERS-active polymer membrane, together with a modified immunoassay method is demonstrated herein, which reduces the overall waiting analysis time (for proteins and other biomolecules as analyte) compared to traditional methods.

The biocompatible SERS-active polymer membrane may also be deemed a SERS sensor having intrinsic anti-bacterial effect due to the presence of silver nanoparticles. For instance, the SERS-active nanoparticles, which can comprise silver nanoparticles as one non-limiting example, can be incorporated on the flexible and porous polymer membrane. Advantageously, such SERS-active silver nanoparticles not only provide for Raman signal enhancement but also confer an anti-bacterial effect, hence further providing a therapeutic benefit.

Moreover, polymeric material that has therapeutic properties can be used as the flexible and porous polymer membrane. Further, a stretchable polymer can be used to form nanostructures in situ as demonstrated in one or more of the examples herein. It is possible to achieve label-free biomarker detection directly from the flexible and porous polymer membrane in combination with data analytics (chemometrics).

Advantageously, biomarkers of small molecules like uric acids and other chemicals can be detected directly using the present biocompatible SERS-active polymer membrane. The present biocompatible SERS-active polymer membrane can be incorporated to be part of wound dressing or band-aids and hence no need for separate sample collection process.

The method of forming the present membrane involves a rapid immobilisation of biomarkers on to metal nanostructures to avoid multistep process (for proteins and other biomolecules as analyte). The present method of forming the present membrane offers a universal approach to create metal nanostructures on the planar and/or flexible surfaces.

The present membrane offers the capability of rendering this platform operable for carrying out multiplexing for multiple biomarker detection. A simple portable Raman reader can be developed that has small footprint for point-of-care (POC) detection, with automated assay performance.

As compared to a method of inkjet printing for forming the SERS-active nanoparticles on a membrane, the present method directly deposits a thin “film” of silver and/or gold layer by means of dry deposition methods (such as e-beam evaporator or sputtering techniques) onto cellulose or polymer membrane. The “film” can comprise separate discrete (i.e. spaced apart and not a continuous layer) structures of the silver and/or gold. Further, the present method provides better uniformity of nanoparticle decoration across the substrate surface, is highly scalable and reproducible compared to traditional inkjet printing method. A large area of coverage is a challenge by means of inkjet printing, which undesirably leads to nanoparticles aggregations and large areas without any nanoparticles. Also, the present method affords easier control of uniform multilayer nanoparticle deposition.

The present biocompatible SERS-active polymer membrane has the following additional advantages. Both cellulose membrane and thin film of elastomeric polymer can be used to deposit uniform nanoparticle thereon by dry deposition technique which is reliable, reproducible and scalable to large area. Regarding its use, a modified immunoassay method has been developed for performing the diagnosis by simplifying the number of steps needed to perform the test in a short turnaround time.

Details of various embodiments of the biocompatible SERS-active polymer membrane and advantages associated with the various embodiments are now described below. Where the embodiments and advantages are demonstrated in the examples further hereinbelow, they shall not be reiterated for brevity.

In various embodiments, the present disclosure provides for a biocompatible SERS-active polymer membrane configured to detect biomarkers in a sample, comprising: a flexible and porous polymer membrane; and SERS-active nanoparticles formed on the flexible and porous polymer membrane, wherein the flexible and porous polymer membrane comprises cellulose or an elastomeric polymer. The term “flexible” herein refers to a material that can be contorted, twisted, and stretched to a reasonable extent without being damaged.

In various embodiments, the elastomeric polymer may comprise a silicon-based elastomer or the elastomeric polymer may comprise polydimethylsiloxane. Said differently, besides PDMS, other silicon based elastomers can be used as the elastomeric polymer. Such elastomeric polymers are advantageously flexible. Flexible elastomeric polymer allows for in situ formation of SERS-active nanostructures (i.e. SERS-active nanoparticles) described later herein under the present method.

In various embodiments, the flexible and porous polymer membrane may be functionalized with an anchoring agent to render an amine functional group or a mercapto functional group for binding to a SERS-active nanoparticle. In various embodiments, the anchoring agent may comprise (3-aminopropyl) triethoxysilane, (3-mercaptopropyl) trimethoxysilane, and/or mercaptopropylsilatrane.

In various embodiments, the SERS-active nanoparticles may comprise silver, gold, and/or copper.

In various embodiments, the SERS-active nanoparticles formed on the flexible and porous polymer membrane may be spaced apart at 150 nm or less, 100 nm or less, 50 nm or less, spaced less than 50 nm apart, etc., and/or the SERS-active nanoparticles formed on the flexible and porous polymer membrane may have a thickness of up to 10 nm, up to 20 nm, up to 30 nm, up to 40 nm, up to 50 nm, up to 60 nm, etc.

In various embodiments, the biomarkers may comprise a matrix metalloproteinase, tumor necrosis factor alpha, and/or an interleukin.

The present disclosure also provides a method of forming the biocompatible SERS-active polymer membrane described in various embodiment of the first aspect. Embodiments and advantages described for the biocompatible SERS-active polymer membrane of the first aspect can be analogously valid for the present method of forming the biocompatible SERS-active polymer membrane subsequently described herein, and vice versa. Where the various embodiments and advantages have already been described above and examples demonstrated herein, they shall not be iterated for brevity.

In various embodiments, the method includes providing a flexible and porous polymer membrane, wherein the flexible and porous polymer membrane can comprise cellulose or an elastomeric polymer, and depositing SERS-active nanoparticles on the flexible and porous polymer membrane. The elastomeric polymer has been described in embodiments of the first aspect and shall not be reiterated for brevity.

In various embodiments, providing the flexible and porous polymer membrane may comprise contacting the flexible and porous polymer membrane with an anchoring agent to have an amine functional group or a mercapto functional group functionalized thereon for binding to a SERS-active nanoparticle.

In various embodiments, the flexible and porous polymer membrane may comprise an elastomeric polymer and the method may further comprise stretching the flexible and porous polymer membrane up to 60%, up to 50%, up to 40%, up to 30%, up to 20%, up to 10%, etc., prior to depositing the SERS-active nanoparticles.

In certain non-limiting embodiments, depositing the SERS-active nanoparticles may comprise placing the flexible and porous polymer membrane into a coater operable to deposit the SERS-active nanoparticles thereon. In certain non-limiting embodiments, depositing the SERS-active nanoparticles may comprise placing the flexible and porous polymer membrane onto an electron beam evaporator to deposit the SERS-active nanoparticles thereon. In certain non-limiting embodiments, depositing the SERS-active nanoparticles may comprise sputtering a precursor of the SERS-active nanoparticles onto the flexible and porous polymer membrane to have the SERS-active nanoparticles formed thereon. The precursor can be any material that is formable into the SERS-active nanoparticles. For example, the precursor may be a gold-based, a silver-based, and/or a copper-based material.

In various embodiments, depositing the SERS-active nanoparticles may be carried out at a deposition current of 10 mA to 20 mA, 10 mA to 15 mA, 15 mA to 20 mA, etc. In various embodiments, depositing the SERS-active nanoparticles may be carried out for a duration of up to 10 s, up to 20 s, up to 30 s, up to 40 s, up to 50 s, up to 60 s. In various embodiments, depositing the SERS-active nanoparticles may comprise depositing the SERS-active nanoparticles at a rate of up to 1 nm/s, up to 2 nm/s, etc.

The present disclosure further provides a method of determining a state of wound healing in a diabetic individual. Embodiments and advantages described for the biocompatible SERS-active polymer membrane of the first aspect and the method of forming the biocompatible SERS-active polymer membrane can be analogously valid for the present method of determining a state of wound healing subsequently described herein, and vice versa. Where the various embodiments and advantages have already been described above and examples demonstrated herein, they shall not be iterated for brevity.

In various embodiments, the method may comprise contacting a sample extracted from a wound of the diabetic individual with a thiolating agent to have one or more biomarkers suspected to be contained in the sample form one or more modified proteins, incubating the biocompatible SERS-active polymer membrane described in various embodiments of the first aspect with the one or more modified proteins to immobilise the one or more modified proteins on SERS-active nanoparticles of the biocompatible SERS-active polymer membrane, providing one or more Raman probes attached with one or more antibodies, wherein each of the one or more antibodies binds specifically to one corresponding modified protein from the one or more modified proteins, contacting the one or more Raman probes with the biocompatible SERS-active polymer membrane having the one or more modified proteins immobilised thereon, and subjecting the biocompatible SERS-active polymer membrane to SERS spectroscopy to generate one or more SERS signals corresponding to the one or more biomarkers, respectively.

In various embodiments, the method may further comprise contacting the biocompatible SERS-active polymer membrane described in various embodiments of the first aspect with a serum albumin to have the serum albumin immobilized on surfaces of the flexible and porous polymer membrane which are not occupied by the one or more modified proteins. In various embodiments, the thiolating agent may comprise a Traut's reagent. The serum albumin can be a bovine serum albumin (BSA) as a non-limiting example.

In various embodiments, the one or more antibodies may comprise an antibody of matrix metalloproteinase, an antibody of tumor necrosis factor alpha, and/or an antibody of interleukin.

To provide a better understanding of the method of determining a state of wound healing, a non-limiting example is described. In various non-limiting examples, biomarkers to be detected from a wound sample can include, for example TNF-α. The sample suspected to contain the biomarkers is mixed with a thiolating agent to form thiolated proteins, which can then be coated onto SERS-active nanoparticles of the biocompatible SERS-active polymer membrane (e.g. a CF membrane of the present disclosure as an example). BSA can then be coated to occupy non-specific binding sites on the biocompatible SERS-active polymer membrane. Antibodies (in this instance anti-TNF-α) can then be attached with a probe, which can then be contacted with the CF membrane having the thiolated proteins for generating a SERS signal from the probe, wherein the antibody (in this instance anti-TNF-α) is specifically bound to corresponding thiolated TNF-α. That is to say, SERS signals are generated when the antibody (in this instance anti-TNF-α) binds to the thiolated TNF-α on the SERS-active nanoparticles of the biocompatible SERS-active polymer membrane.

In summary, the surface geometrical characteristics of cellulose fibers (as a non-limiting example) was exploited to fabricate CF-based SERS substrates by depositing SERS-active (e.g. Ag) particles onto chromatography CF substrates, and the resulting functionalized CF was used to detect and quantitate protein biomarkers present in wound fluids, e.g. MMP-9. TNF-α, IL1-α and IL1-β. CF substrates, as one non-limiting examples of the present biocompatible SERS-active polymer membrane, offer several advantages over traditional substrates, being flexible, economical, easy to use, and with uncomplicated analyte uptake (self-wicking). These properties are ideal to facilitate the biocompatible SERS-active polymer membrane (e.g. CF sensors) to be incorporated into existing wound dressing materials and formats. The sensing strategy of the biocompatible SERS-active polymer membrane (e.g. CF membrane) as SERS sensors is based on the strong adsorption of plasmonic metal layers onto the CF surface. The present disclosure also describes for the methodology of fabrication and configuration of the biocompatible SERS-active polymer membrane (e.g. CF substrates) for SERS enhancement, conditioning of Ag deposition variables and stabilizing the Ag coating with, for example, ATPES. The sensor prototypes herein demonstrated linear responses within biologically relevant concentrations, ranging from 10 to 500 ng/ml for MMP-9, and in the case of TNF-α, non-linear response for the range of 5 to 100 ng/ml. In addition, the present disclosure also demonstrated a different range of linear responses for IL1-α and IL1-β. By achieving the highest detection point of sub-μg/mL for these biomarker proteins, the biocompatible SERS-active polymer membrane (e.g. flexible CF substrate) reveals the effectiveness, specificity and high sensitivity required to detect and measure clinically relevant changes in critical biomarkers evident in the wound environment. The prototype SERS sensors described herein, possess considerable advantages for permitting non-invasive monitoring of chronic wounds, adding to the armamentarium of much needed clinical decision-making tools.

The word “substantially” does not exclude “completely” e.g. a composition which is “substantially free” from Y may be completely free from Y. Where necessary, the word “substantially” may be omitted from the definition of the present disclosure.

In the context of various embodiments, the articles “a”, “an” and “the” as used with regard to a feature or element include a reference to one or more of the features or elements.

In the context of various embodiments, the punctuation “˜”, term “about” and “approximately”, as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### EXAMPLES

The present disclosure relates to a biocompatible SERS-active polymer membrane, its method of forming and its uses.

Traditional protein detection methods include protein separation and purification. Examples of such techniques include, two-dimensional gel electrophoresis, high performance liquid chromatography, digestion by enzyme, and mass spectroscopy. Mass spectroscopy techniques require bioinformatics to predict protein sequences and its three-dimensional structure. Detection of proteins by their function involves protein-ligand interactions-chemiluminescence and fluorescence-based methods may be commonly used for this technique. The disadvantages of these methods include being time consuming, labour intensive and inefficient for high sensitivity and selectivity of protein detection. Fluorophores show broad emission spectra which makes multiplexing protein detection difficult, or even impossible. Also, photo bleaching which may occur with fluorescence-based methods, is an imminent problem that considerably reduces detection limits of fluorescence-based methods.

Herein describes adopting Surface enhanced Raman Spectroscopy (SERS), together with a polymer membrane (e.g. a cellulose membrane) as a substrate, for use in detecting biomarkers to evaluate the progress in wound healing, e.g. diabetes mellitus (DM) related impaired wound healing. SERS exploits the enhancement of Raman scattering by molecules physically or chemically adsorbed onto a substrate. This modality is highly sensitive and specific for chemical and bio-molecular detection as it produces unique vibrational fingerprint spectra of individual molecules and their characteristics.

A variety of SERS substrates can be fabricated with different structural morphologies, including electrochemically roughened electrodes, colloidal nanoparticles films, metal film nanospheres (MFON), metal islands, electron beam lithography regular plasmonic mircoarray and nanoholes. Traditionally, silicon, glass and glass fibers appear to be common SERS substrate as they render low SERS background signals and ensure that most analyte attach to the substrate. However, these substrates tend to be not ideal for routine point-of-care purposes due to their fragility and rigidity. The studies herein explore cellulose membrane as an alternative for the substrate since it is cheap, extensively available and can be readily recyclable. Other advantages of using cellulose membrane as substrate include its natural hydrophilicity and its wicking ability that allows it to drive sample flow without depending on mechanical intervention. Plasmonic cellulose membrane can be made through various methods, such as soaking, inkjet printing, screen printing, deposition and filtration, and in a variety of forms such as microarrays, swabs and dipsticks. The present biocompatible SERS-active polymer membrane includes a flexible and porous polymer membrane that acts as a substrate incorporated with SERS-active nanoparticles, which afford reproducible signals for evaluating progress of wound healing, e.g. TNF-α biomarkers signals for the evaluation of the progress of DM related impaired wound healing.

Diabetes causes acute complications such as hyperosmolar hyperglycaemic state, diabetic ketoacidosis or death and chronic complications such as ulcers on the foot, eye damage, kidney, and cardiovascular diseases. 2 to 5% of diabetic individuals can develop gangrene and/or ulcers on their foot, of which 15% may have to undergo amputation. In order to alleviate these problems, disease and wound management may have to be started early. In this connection, the traditional protein detection methods mentioned above face several drawbacks for use in disease and wound management as they tend to be, for example, highly time consuming, labour intensive, inefficient for high sensitivity and selectivity of protein detection, and susceptible to poor detection limit. A analytical biochemistry assay may have been developed to circumvent this, which is the enzyme-linked immunosorbent assay (ELISA). ELISA is deemed a method to detect a protein dissolved in a liquid using antibodies that can bind the proteins. Although ELISA may be used for protein detection, it inevitably has some drawbacks. The ELISA procedure is also time consuming with incubation steps that may require 2 hours to overnight, or even longer. ELISA is also laborious with many steps and the biomolecule recognition sensitivity may be insufficient.

On the other hand, the present biocompatible SERS-active polymer membrane, its method of forming and its uses, involves SERS for detecting proteins, including biomarkers. Advantageously, the biomarkers can be detected using SERS with or without additional labeling. Each molecule renders a unique Raman spectrum, i.e. a characteristic fingerprint specific to the molecule. Further advantageously, the present biocompatible SERS-active polymer membrane, its method of forming and its uses, are rapid, produces high resolution results and needs only a single excitation wavelength. Multiplexing and simultaneous detection of multiple biomarkers and/or proteins at the same time is also possible.

Traditionally, a SERS substrate may have nanometer-sized metal structures fabricated on silicon wafers. Fabrication of these substrates tend to be laborious, expensive and require a cleanroom facility. Also, silicon substrates cannot be used directly on patients to collect wound biomarkers that are present in trace amounts because they are fragile and rigid. The present biocompatible SERS-active polymer membrane is a flexible and easy to fabricate membrane-based SERS substrates that addresses this limitation. The present biocompatible SERS-active polymer membrane can be used in wound exudate collection and diagnosis. Since SERS is a surface sensitive technique, it tends to require a planar surface, which the present biocompatible SERS-active polymer membrane offers and hence highly compatible with SERS. As one example, the biocompatible SERS-active polymer membrane can include a cellulose fiber (CF) based SERS membrane that can conduct the fluid through their porous membrane structure and act like a lateral flow strip. In another example. polydimethylsiloxane (PDMS) based flexible SERS membrane is easy to fabricate, low-cost and does not require a cleanroom facility. PDMS is transparent and breathable that allows exchange of gas molecules, rendering it usable on patients directly to collect the wound biomarkers. PDMS is also non-toxic. PDMS SERS membrane can be manufactured in any shape, size, or thickness and thus it is a very versatile. PDMS based SERS membrane are clastic and can return to its original form even after undergoing contortions, also wickable.

The present biocompatible SERS-active polymer membrane and its method of forming and uses are described in further details, by way of non-limiting examples, as set forth below.

### Example 1: General Introduction of Biocompatible SERS-Active Polymer Membrane

Wound healing involves multifaceted coordination of cellular responses to the presence of protein mediators and regulators, and mobilization biological events to facilitate tissue repair. In the presence of pathophysiological abnormalities, such as high blood sugar level in diabetes, this coordination tends to fail, impairing wound healing and tissue repair. Traditional approaches to managing chronic wounds tend to remain reliant on regular monitoring with clinical reviews over prolonged periods of time. This tends to be complex, tedious, and labor intensive, giving rise to inefficiencies, high costs, and often poor compliance, ensuring chronic wound management is among the most impactful current socioeconomic healthcare challenges.

Reported clinical reviews include capturing digital photographs of the wound and physically measuring its dimensions to monitor the wound size over time; little information is captured from the wound at the cellular or tissue level. Due to the complexity of the mechanisms involved in wound healing, there potentially exists a plethora of possible biological targets and markers, including inflammatory cytokines, growth factors, and proteinases/proteinase inhibitors associated with wound healing and tissue repair.

Understanding the mechanisms that impair wound healing helps identify potential predictive, diagnostic and indicative biomarkers and determined their role in the wound healing cascade of events. The biomarkers present in the wound fluid, tissue specimens or serum, have potential to grant predictive outcomes for personalized treatment, reveal the wound healing status and response to treatment, and facilitate a more informed clinical assessment.

Traditionally, a multi-omics approach to evaluating putative biomarkers associated with disease involves collecting biopsy samples from patients and transferring these to a laboratory for quantification using specialist techniques. For example, enzyme-linked immunosorbent assays (ELISAs) or liquid chromatography-mass spectrometry (LC-MS). This adds to lengthy analytical procedures as clinical samples have to be prepared in accordance with the assays employed. Traditional ELISAs may be sufficiently sensitive to determine up to 1 pg/mL for some protein biomarkers but they are laborious, costly and not suitable for point-of-care (POC) detection of multiple biomarkers. Also, LC-MS may have the capability to detect multiple biomarkers with very high sensitivity and specificity but it is time consuming. involves expensive equipment and hence is not feasible for POC analyses.

The present biocompatible SERS-active polymer membrane (also termed “sensor” herein for brevity), which is incorporable/usable as sensors for clinically relevant indicators on wound dressings, offers a convenient non-invasive advantage to sample wounds directly, improving clinical measures and informing clinical decisions. This may have a direct impact on patients, optimizing the clinical workflow and improving the management of individuals with chronic wounds. Through thoughtful investigation, the sensor developed herein can simultaneously detect multiple biomarkers, be portable, flexible and compatible with dressings, and importantly be cost-effective.

The present biocompatible SERS-active polymer membrane, in one non-limiting example, couples cellulose fiber (CF) membrane with surface enhanced Raman spectroscopy (SERS) to detect biomarkers associated with impaired wound healing. SERS exploits the enhancement of Raman scattering by molecules physically or chemically adsorbed onto a substrate (e.g. nanoparticles). This modality is highly sensitive and specific for chemical and bio-molecular detection as it produces unique vibrational spectra (“fingerprint”) for individual molecular species. A selection of other SERS substrates have been fabricated with different structural morphologies, including electrochemically roughened electrodes, colloidal nanoparticles films, metal film nanospheres (MFON), metal islands, electrospinning nanofibrous, tunable SERS substrates, silicon based nanogap and nanopillars, electron beam lithography regular plasmonic micro-array and nanoholes.

Silicon and glass may be traditionally used as SERS substrates as they yield low background signals and ensure that most analytes attach to the substrates (e.g. nanoparticles). However, silicon and glass are less than ideal for incorporation into wound dressings, being undesirably fragile and rigid. In the examples of the present disclosure, studies have explored using alternative substrates, including CF-based paper substrate. CF is cheap, plentiful and readily recycled. Other advantages of using CF as a substrate, include its natural hydrophilicity and its wicking ability that allows it to drive sample flow without external input. Further, plasmonic CF substrate can be made through various methods, such as soaking, inkjet printing, deposition and filtration, and in a variety of form factors such as microarrays, swabs and dipsticks.

Cutaneous wound healing is characterized as a series of overlapping cascading events. Wounds in which any of these events is perturbed, exhibit delayed healing and commonly remain ‘stuck’ in the inflammatory phase. The pro-inflammatory cytokines tumor necrosis factor alpha (TNF-α) and interleukin-1 (e.g. IL1-α and IL1-β) are elevated in patients with chronic wounds. High levels of activated MMP-2 and MMP-9 are also present in chronic wounds and hinder wound closure and healing. These data aid the selection of TNF-α, IL1-α, IL1-β, and MMP-9 for demonstrating in the examples as candidate biomarkers to be incorporated in the present prototype CF-based SERS biosensors. In various examples herein, flexible, cost-effective, reliable, easy-to-fabricate plasmonic SERS substrates are developed using flexible CF membrane. The plasmonic silver nanoparticles, which are non-spherical in shape were developed and decorated onto the CF membranes using dry deposition by sputtering method. The resultant affordable SERS platform was used to detect MMP-9, TNF-α, IL1-α and IL1-β in simulated wound fluids in the clinically relevant nM to pM range. It is envisioned that these substrates after activation can be incorporated into wound dressings for the routine monitoring of wound biomarkers. This proof-of-concept study illustrates the potential application of SERS biosensors to predict the healing status of wounds and monitor healing trajectory in a non-invasive manner.

### Example 2: Materials and Methods

Materials used in the examples herein include (3-aminopropyl) triethoxysilane (APTES) (Sigma Aldrich), 2-iminothiolane hydrochloride (Traut's reagent) (Sigma Aldrich), 1-ethyl-3-(3-dimethylaminopropyl) carbodiimide (EDC) (Thermo Fisher Scientific), N-hydroxysuccinimide (NHS) (Thermo Fisher Scientific), 4-aminothiophenol (4-ATP) (Sigma Aldrich), 2-naphthalenethiol (2-NT) (Sigma Aldrich), recombinant human TNF-α protein (TNF-α) (Abcam), anti-TNF-α antibody (anti-TNF-α) (Abcam), recombinant human MMP-9 protein (Abcam), anti-MMP-9 antibody (Abcam), recombinant human IL1-α protein (Abcam), anti-IL1-α antibody (Abcam), recombinant human IL1-β protein (Abcam), rabbit polyclonal anti-IL1-β antibody (Abcam), human MMP9 ELISA Kit-ab246539 (Abcam), bovine serum albumin (BSA) (Sigma Aldrich), ethanol (Merck) and phosphate buffered saline (PBS) (Lonza) were purchased as indicated. Sylgard 184 Silicone Elastomer Kit, polydimethylsiloxane (PDMS) from Dow Corning, Whatman Grade 1, 2.5 cm cellulose membranes from GE Healthcare Life Sciences. (3-mercaptopropyl) trimethoxysilane (MPTMS), mercaptopropylsilatrane (MPS), bovine serum albumin (BSA), and ethylenediaminetetraacetic acid (EDTA), were purchased from Sigma Aldrich.

### Example 3: Preparation of CF Substrates

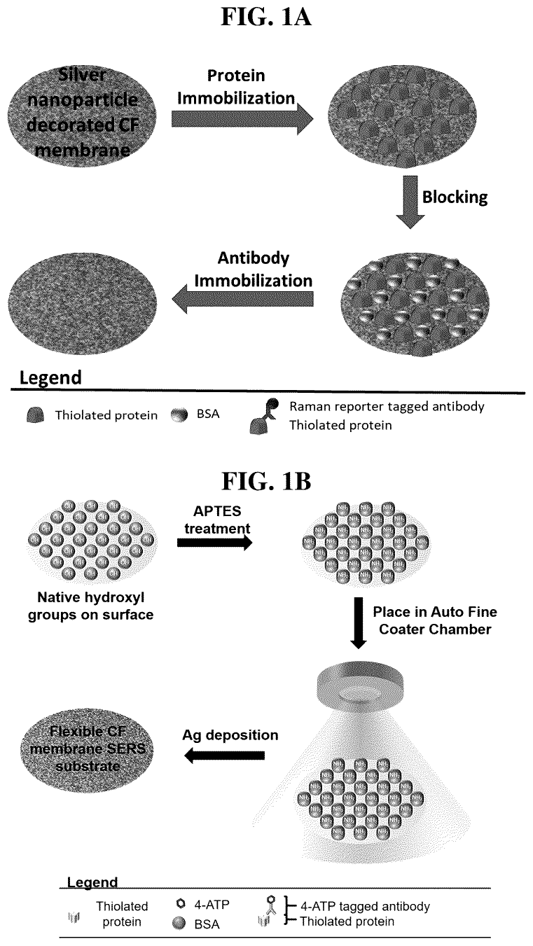

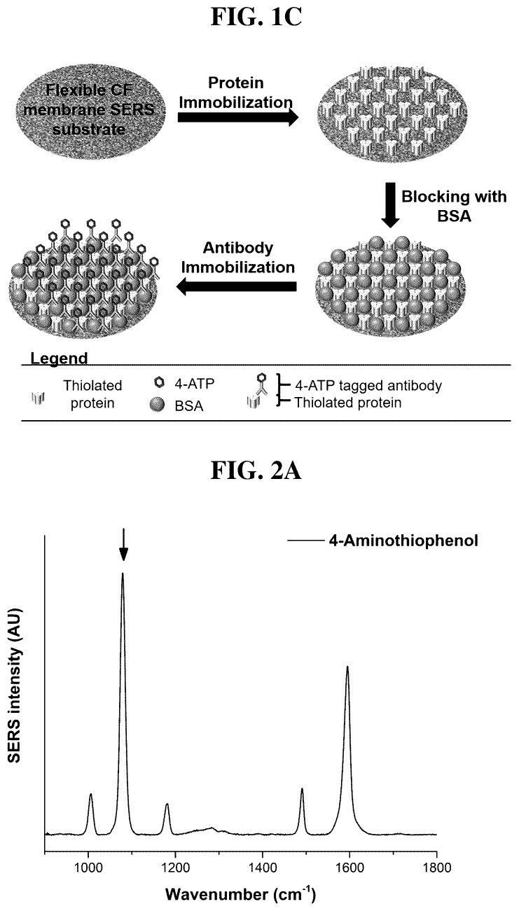

In general, 2% APTES (or MPTMS or MPS) in ethanol were used to incubate Whatman Grade 1, 2.5 cm cellulose membranes (GE Healthcare Life Sciences) or cellulose fibers on a planar surface and left to dry in vacuum desiccator. The same protocol was used for PDMS. The APTES (and MPTMS and MPS) serves as anchoring agents for performing silanization onto a hydrophilic surface of a substrate and a mercapto group can be used to form a self-assembled monolayer and subsequently improve the efficiency of immobilisation with silver and gold colloid by means of strong metal-ionic bonding. In various non-limiting instances, one type of anchoring agent (i.e. APTES or MPTMS or MPS) is used. In certain non-limiting instances, more than one type of anchoring agent can be used. The cellulose membrane was then washed with acetone, left to dry, and coated with a silver layer (˜50 to 80 nm thickness) using a sputtering/e-beam evaporator system (sufficient to make random nano-islands of silver nanoparticles). After that, the membrane substrates were washed with water and ethanol thrice and left to dry in a desiccator for 1 hr. The resultant flexible SERS membrane where checked for possible background signals and contamination. The fabrication flow of the flexible SERS cellulose membrane is shown in FIG. 1A. Only SERS membrane with no signs of contamination were used for further study.

In more detail, the preparation of CF substrates is illustrated in FIG. 1B. APTES, as a non-limiting example, (300 μl of 2%) in ethanol was added to Whatman (Grade 1) 2.5 cm diameter CF membrane filter (GE Healthcare Life Sciences) and left to dry for 5 mins (e.g. in a vacuum dessicator). The CF substrates were then washed with acetone, left to dry and sputter-coated with Ag (99.999% purity, JEOL) at 10-20 mA (JEOL, JFC-1600 Auto fine coater). The Ag metal layer was deposited at a rate of ˜1 nm/s to achieve different metal thickness. The size of the particle is proportional to the duration of deposition. Here, the deposition condition was configured to form a reproducible particle size of Ag nanoparticles on substrate. Following that, the CF substrates were washed twice with water and ethanol and dried in a desiccator for 1 hr. SERS measurements were performed on the CF substrates to check for background signals and contamination. Only CF substrates with no evidence of contamination were used for biosensor measurements.

### Example 4A: Characterizations

Field emission scanning electron microscope (FE-SEM) from JEOL, was used to image the SERS CF substrates. SERS measurements of samples were performed using a Renishaw InVia Raman upright microscope (Renishaw In Via, UK) with a 785 nm laser. This Raman system was integrated with a Leica microscope and the laser light was coupled through an objective lens (50 x, 0.75 N.A.), which was used to excite the sample and to collect the scattered Raman signal. Prominent Rayleigh scattering was blocked using a notch filter. The beam spot illuminating the sample was ˜1 μm or ˜2 μm. Spectra (10) were acquired from more than 10 different spot areas on each sample. Each spectrum was integrated for 10 s, in the range of 700-1800 cm⁻¹. Post collection processing of the SERS spectra was performed using the WiRE™ v3.4 software associated with the instrument. Multiple spectral acquicision by means of SERS mapping with 100-150 data points spaced out by 5 μm along X and Y axis. The background subtraction was performed by cubic spine interpolation. The instrument was calibrated at 520 cm⁻¹ from standard silicon.

### Example 4B: Immunoassay Procedure for CF Membrane SERS

1 μg/ml. 0.1 μg/ml. 0.01 μg/ml, of modified TNF-α protein solution was obtained via serial dilution of modified TNF-α protein using PBS buffer, 20 μl of modified protein was added to the cellulose membrane substrate and allowed to incubate for 30 mins. After which, the cellulose membrane substrate was washed with 50 μl of PBS buffer thrice and then blocked with 50 μl of 0.5 mg/ml thiolated BSA solution for 30 mins. The cellulose membrane substrate was then washed with 50 μl of PBS buffer twice and checked for any contamination. After that, the cellulose membrane substrates were incubated with 20 μl of 0.1 mg/ml anti-TNF-α-4-ATP solution for 30 mins. The cellulose membrane substrates were washed with PBS buffer twice and left to dry before taking SERS measurements.

### Example 5: Preparation of Modified Wound Biomarker Protein and Antibody

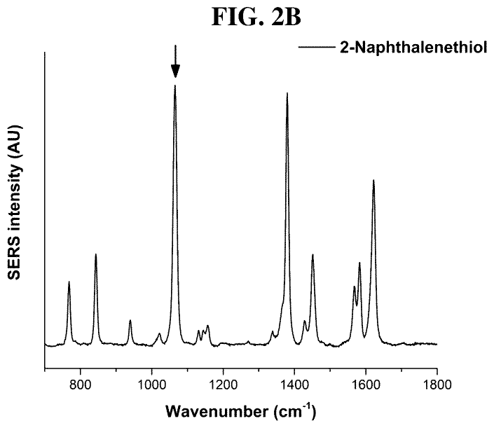

Commercially acquired preparations of MMP-9, TNF-α, IL1-α and IL1-β proteins and antibodies were used as received. 4-ATP and 2-NT were used as the Raman active molecules in this study to estimate the performance of the fabricated substrate. Representative SERS spectra of the Raman reporter molecules were shown in FIG. 2A and 2B. TNF-α protein (10 μg/mL, Abcam) was modified with 14 mM Traut's reagent and filtered using Ultra-0.5 Centrifugal Filter Unit (Amicon) to remove excess Traut's reagent. EDC (400 μL of 50 mM) was added to 100 μL of 1 mg/mL anti-TNF-α solution and incubated for 5 mins. NHS (400 μL of 10 mM) was then added to the mixture and left to incubate for 5 mins. Next, 100 μL of 1 mM 4-ATP was added to the EDC-NHS anti-TNF-α solution and left to incubate for 30 mins. The solution was subsequently filtered to remove excess unreacted reagents and the resultant protein filtrate was reconstituted to 0.1 mg/mL anti-TNF-α modified with 4-ATP (anti-TNF-α-4-ATP). This protocol can be adapted or used for other protein biomarkers and its corresponding antibody and capturing agent.

### Example 6: Preparation of CF Substrate for Biomarker Sensing

The method of preparing CF substrates for SERS biomarker sensing is presented, as a non-limiting example, in FIG. 1C. Protein modification via Traut's reagent was employed to minimize non-specific binding on the substrate surface that could be introduced with every incubation step, improving the experimental workflow. Traut's reagent was used for the thiolation of primary amines of commercially acquired preparations of MMP-9, TNF-α, IL1-α and IL1-β proteins, introducing sulfhydryl groups while retaining the charge properties of the original amino group. Thiolated proteins were filtered to remove excess unreacted Traut's reagent and serially diluted in PBS to yield final concentrations of 5 to 5000 ng/mL. These were used to establish the limit of detection of the CF substrates. Modified proteins of varying concentrations were immobilized onto Ag-coated CF substrates by incubation with thiolated protein solutions. Samples were washed thoroughly with PBS buffer to remove weakly immobilised and/or unbound thiolated proteins. Following the immobilisation of the thiolated proteins and PBS washing, the CF substrates were incubated in a solution of 0.5 mg/mL BSA solution to occupy non-specific binding sites on the CF substrate surface. Exposure to the corresponding MMP-9. TNF-α, IL1-α and IL1-β antibodies tagged with 4-ATP solution (Raman reporter) resulted in specific binding to thiolated MMP-9. TNF-α, IL1-α and IL1-β proteins respectively, detected as 4-ATP SERS Raman spectra.

### Example 7: Results and Discussion-Design and Fabrication of Flexible CF Membrane Substrates

The fabrication process of the flexible CF SERS substrates is depicted in FIG. 1A and 1B. Firstly, the CF-based membrane surface was washed with pure water (Milli-Q) followed by cleaning with ethanol and acetone to remove any residual organic impurities that may have adhered to the membrane and was then dried. In order to improve the adhesion of the metal nanoparticles onto the CF-based membrane surface. the APTES linker molecules were attached by means of self-assembled monolayer (SAM) onto the whole CF membrane surface. This was achieved by conjugating the hydroxyl functional group present in the CF membrane surface with that of ethoxy group present in APTES. The presence of free amine group from APTES binds the Ag nanoparticles strongly to the CF membrane substrate during the sputtering stage. In the present method, a sputtering technique was used to decorate single layer nanoparticles uniformly on the whole area of the substrate surface. Prior to using the SERS substrate, unbound and loosely bound Ag nanoparticles were removed by repeatedly washing the CF membrane surface, to ensure reliable and robust CF based SERS substrates.

### Example 8: Results and Discussion-FE-SEM Characterization of Flexible CF Membrane Substrate

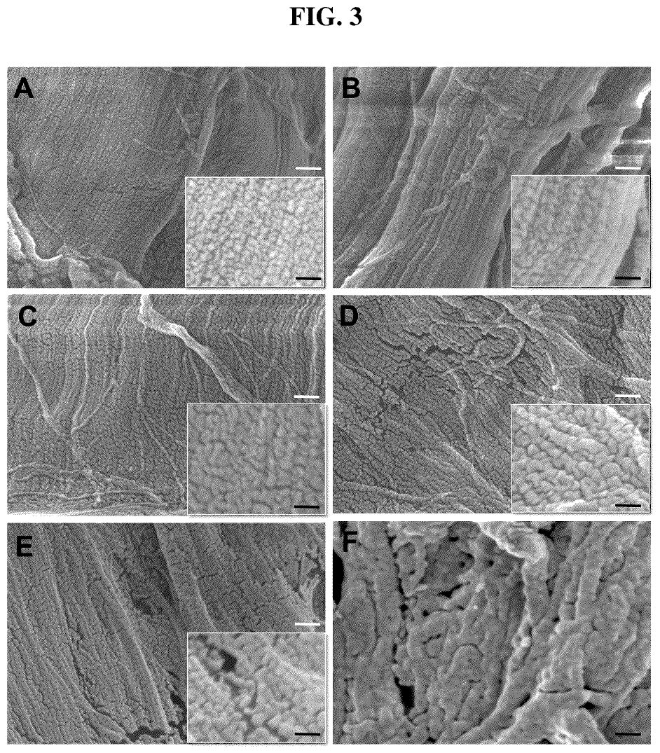

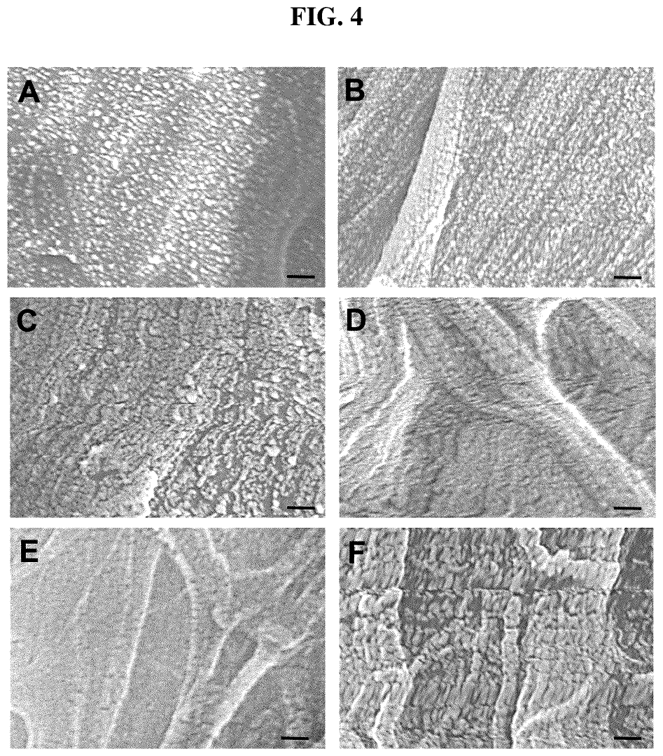

The microstructure of the CF-based substrates resulting from varying the Ag deposition conditions, including the current and deposition time, were investigated using FE-SEM. FIG. 3A to 3F show a multilayer morphology of Ag particles for the substrate prepared at 10 mA with deposition times varied from 30 to 80 seconds. It was observed that the distribution of the Ag particles became increasingly disordered with longer sputter-coating times. Similarly, FIG. 4A to 4F depict the morphology of the Ag deposition at 20 mA with deposition times varying from 20 to 80 seconds. Increasing the sputtering current increased the deposition rate of the Ag and coincidently prevented the aggregation of Ag particles before deposition, suggested by the increased Ag layer thickness with longer deposition times without evidence for more Ag aggregates. To obtain high Raman signal enhancement, the gap between Ag nanoparticles should be less than 50 nm as the smaller the gap, the larger the SERS enhancement. It is observed that as the thickness of the Ag layer increased, the gap between nanoparticles decreased, up to 60 seconds of Ag deposition. With longer deposition time, it was evident nanoparticles fused together, resulting in weaker electromagnetic fields and loss of plasmonic properties.

### Example 9: Results and Discussion-Evaluating Performance of Flexible CF Membrane Substrate With 2-NT Raman Probe

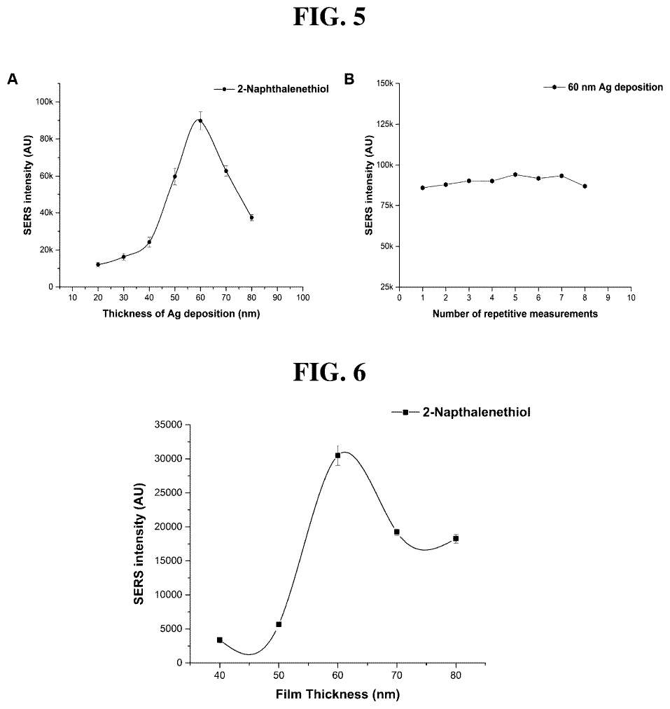

CF membranes decorated with varying thickness of Ag nanoparticles were tested with the Raman active molecule 2-NT to optimize fabrication of the CF-based SERS substrate. FIG. 2A and 2B illustrate a representative 4-ATP and 2-NT SERS spectrum, respectively. The SERS properties of this Raman probe were studied under 785 nm laser excitation-the characteristic SERS band for 4-ATP and 2-NT is evident at 1066 cm⁻¹, annotated with an arrow. FIG. 5A demonstrates that increasing the duration of Ag deposition coincidently increases SERS enhancement, however, this correlation was only observed in samples with Ag layers with thicknesses up to approximately 60 nm. The rate of deposition of Ag was calculated to be approximately 1 nm/s. When the duration of Ag deposition is increased beyond 60 seconds, SERS enhancement is weakened, corresponding with nanoparticles fusing and losing optimal spatial distancing. To characterize the Ag-coating uniformity, SERS measurements were acquired across different regions of the substrate (FIG. 5B). The overall variation of measures was found to be less than 5% and within the acceptable range. This was interpreted as an indication that the Ag nanoparticles were uniformly decorated around the CF. In FIG. 6, the SERS intensity of 2-NT illustrates the variable thickness of Ag particle deposition obtained with 10 mA current. When the Ag deposition was performed using 10 mA current, it was found that the maximum SERS enhancement was lower than that obtained using 20 mA current for Ag deposition. This was interpreted as an indication of Ag nanoparticles deposited at 10 mA aggregate and attenuate SERS signals. Among the substrates tested, CF-based substrates sputtered at 20 mA for 60 seconds yielded the highest 2-NT SERS signal intensity at 1066 cm⁻¹. Thus, depositing Ag onto CF membranes at 20 mA for 60 seconds was selected as the condition for sputter-coating examples.

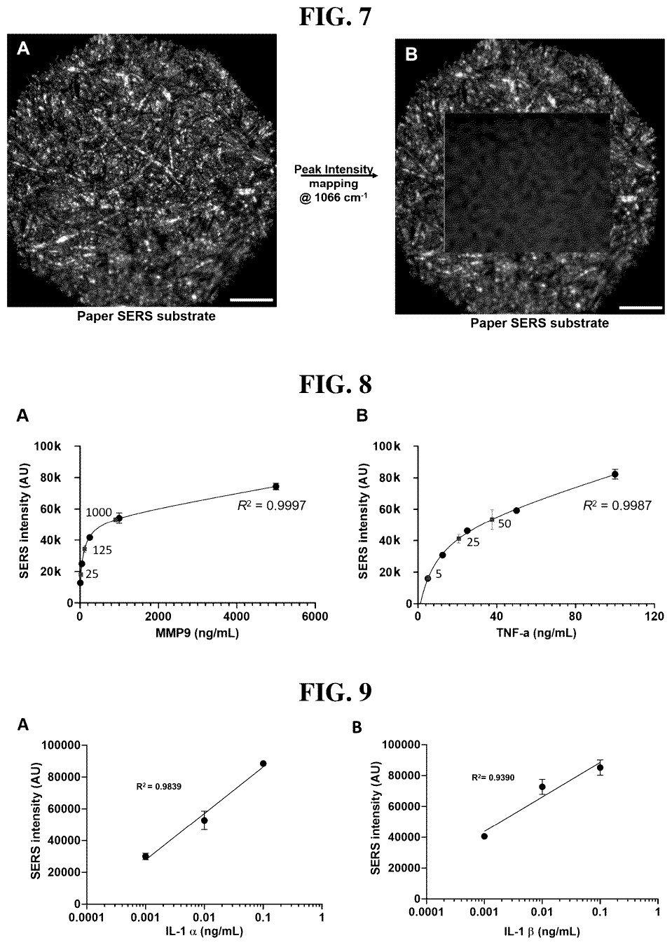

SERS mapping was also performed using the above CF substrate as a non-limiting example to acquire large area scanning to demonstrate the uniformity of signal enhancement along the CF membrane fibers. For this, the substrate's surface was scanned to obtain the 2-NT signal intensity at 1066 cm⁻¹ using the 5× magnification objective lens. FIG. 7A and 7B show the optical image of the substrate surface with overlaid SERS signal spatial map in red heat map. The bright red color corresponds to high SERS enhancement region while black color corresponds to weak or no SERS enhancement region due to absence of nanostructures in the SERS heat map. As shown in FIG. 7A and 7B, it is observed that uniform SERS signal for 2-NT on the membrane surface along the length of the CF fibers while poor or no SERS signal between the intertwining CF fibers was detected. This demonstrates the uniformity of the CF membrane, making it a potentially good candidate as a flexible SERS substrate.

### Example 10: Results and Discussion-Detection of MMP-9, TNF-α, IL1-α and IL1-β With CF-Based SERS Substrates

Selected proteases and interleukins associated with the wound healing response and reported to be elevated in chronic wounds were tested using a modified immunoassay method with the present fabricated CF-based SERS substrate. The examples involve use of the CF-based SERS substrate to characterize wound exudates via 4-ATP as the Raman probe conjugated to anti-MMP-9 and anti-TNF-α immunoglobulins. Said differently, in such non-limiting examples the 4-ATP is immobilized to the antibody. FIG. 8A and 8B depict the relationship between analyte concentration (pg to μg/mL range) and intensity of the SERS signal at the 1078 cm⁻¹ peak, corresponding to the Raman probe 4-ATP for each analyte (i.e. MMP-9 and TNF-α). SERS sensitivity to MMP-9 over concentrations from 10 ng/ml to 5000 ng/ml was tested. The calibration plot was observed to exhibit concentration dependent linear correlation for MMP-9 only up to 500 ng/mL, with R² value of 0.952; beyond which no linear correlation was observed. In the case of TNF-α, on the other hand, a lower concentration range of 5 ng/mL to 100 ng/mL was tested. The resultant calibration plot yielded a non-linear correlation over the whole concentration range with a R² value of 0.988. Measured results for the cytokines IL1-α and IL1-β are shown in FIG. 9A and 9B, respectively. These data demonstrate achievement for detection of these target protein species with the present flexible CF SERS substrates within the biologically relevant, sub-ng/mL concentration range. Notably, the CF SERS detection of the target bioanalytes is more sensitive than data reported for an equivalent nano-mechanical sensor, and for traditional sandwich immunoassays. The levels of IL-1 and TNF-α reported in healing wounds are ˜1 ng/ml; in chronic wounds, these levels commonly reach 50 and 15 ng/ml, respectively. The wide detection range of the CF SERS substrate is advantageous for determining trace levels of biomarkers targets in the wound environment during healing. Further substrate surface functionalization can help expand the range of detection. The maximum detectable concentration range of traditional ELISA is less than the present biocompatible SERS-active polymer membrane (e.g. the functionalized CF-based SERS substrate). From a manufacturing and reproducibility perspective, the performance of the CF-based substrates was found to be relatively uniform across the entire active substrate surface. The notable sensitivity of the present approach may be attributed to these characteristics: (1) the Ag layer structure allows decoration of ATP binding sites, thus enhancing detection performance significantly; (2) the deposition of Ag nanoparticles greatly enlarged the active surface area available in the sensor, ensuring it effectively captures higher concentrations of the target biomarker. It is established herein a reliable and scalable method to manufacture flexible plasmonic substrates using CF.

### Example 11: Results and Discussion-Detection of Spiked Concentrations of TNF-α & MMP-9 Proteins

To validate the specificity and efficiency of detecting the target biomarkers in complex biological fluids, a proof-of-concept study was performed in which the analyte of interest was spiked into samples containing serum albumin (e.g. BSA) and other plasma-derived species to imitate the actual biological condition. In order to achieve this, MMP-9 and TNF-α proteins were spiked into a solution of 0.5 mg/mL BSA in phosphate buffered saline, pH 7.4. In order to simulate serum, the proteins in the resultant spiked solutions were then thiolated using Traut's reagent. To study the feasibility of CF-based SERS platform (as one of the examples) in real applications, sample vials spiked with MMP-9 and TNF-α together were prepared with desired analyte concentrations (25, 125 and 1000 ng/ml and 5, 25 and 50 ng/ml, respectively) with above mentioned method. FIG. 8A and 8B illustrate the detection of spiked concentrations of MMP-9 and TNF-α biomarkers (square points), respectively, by interpolating the detected concentrations of the analytes from the respective SERS calibration plot obtained at 1078 cm⁻¹. In the case of MMP-9, an accuracy of more than 95% was obtained for lower concentrations of 25 and 125 ng/mL, due to the linear range of the calibration plot up to ˜500 ng/ml (FIG. 8A). Beyond this concentration range, the calibration plot was no longer linear, resulting in reduced accuracy of the SERS sensor to 90.9% for concentrations above 500 ng/mL. Hence, the concentration ranges in which the CF-based SERS membrane biosensor tend to exhibit highest accuracy is from 5 ng/mL to 500 ng/ml. Similarly, in the case of the TNF-α biomarker, linear correlation in the calibration plot was observed from the concentration ranges of 5 to 25 ng/ml, after which the specificity of the sensitivity of the substrate decreased to 75% at concentration of 50 ng/mL (FIG. 8B). Therefore, solutions with biomarker concentrations beyond 50 ng/ml warrant further dilution to detect with high accuracy.

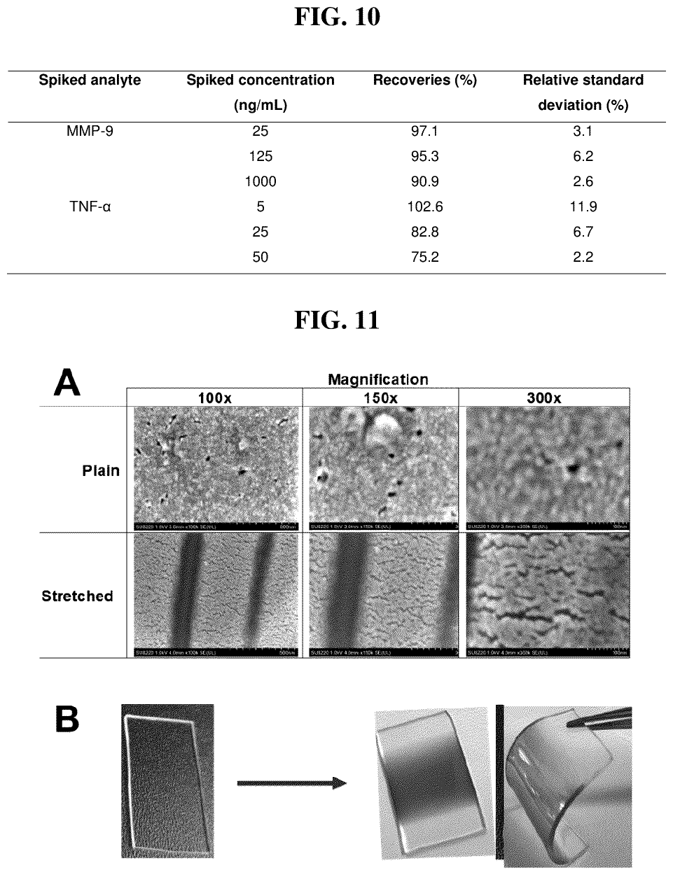

As shown in FIG. 10, recoveries of MMP-9 in spiked samples ranges from 90 to 97% with a relative standard deviation of <6.2%, while that of TNF-α varies from 75 to 102% with a relative standard deviation of <11.2%. This suggests that the recovery of MMP-9 is more efficient compared to that of TNF-α protein. This could be ascribed to the better correlation of SERS intensity obtained with different and a large range of concentrations for MMP-9, i.e. R² value closer to 1 whereas in the case of TNF-α, the saturation phase of the calibration plot had not yet been achieved.

### Example 12: Method-Fabrication and Characterization of Polydimethylsiloxane (PDMS) Substrate

As mentioned in example 3, a PDMS substrate can be modified in the same manner as the CF substrate. Prior to that, a PDMS membrane can be fabricated as follows.

PDMS monomer and a curing agent was mixed in a container in 10:1 ratio. Mixture was spread in a flat container. The PDMS in the petri dishes were subjected to alternating vacuum and room pressure to remove any trapped air bubbles. The PDMS was cured overnight at 65° C., cooled to room temperature and stored wrapped in parafilm. A 25 mm by 45 mm rectangle was cut out and immersed in 100% ethanol, sonicated for 30 mins, and dried to remove small oligomers that are not crosslinked in the elastomeric matrix. The resultant PDMS rectangle was treated with oxygen plasma for 1 min. The PDMS was immersed in 2-5% (3-aminopropyl) tricthoxysilane in ethanol for 10-30 minutes, rinsed with ethanol and dried.

In one approach—the PDMS rectangle was placed in the stretching device and stretched 15-20 mm (50-60% stretching). Stretched PDMS with device was placed in a JEOL JFC-1600 Auto-fine Coater or onto the e-beam evaporator system that was loaded with a silver precursor for depositing silver thereon. The precursor is formable into SERS-active nanoparticles. The precursor can be, for example, a silver-based material, a gold-based material, and/or a copper-based material. In this example, silver was coated on the PDMS to a thickness of 50-75 nm at 20 mA. The idea here is to make plasmonic metal nano-islands that do not form into a continuous metal film. The stretched PDMS was released which results in silver and/or other plasmonic metal nanoparticles that are closely arranged resulting in very high enhancement factor. A 3 to 5 mm by 3 to 5 mm squares were cut and used for subsequent procedures.

In a second approach—the PDMS rectangle was placed onto a glass slide and JFC-1600 Auto-fine Coater loaded with a silver target was used, or the PDMS rectangle was placed onto the e-beam evaporator system loaded with a silver target. Silver and/or other plasmonic metal was coated on PDMS to a thickness of 75-100 nm at 20 mA. This is to get continuous metal film. This metal coated PDMS rectangle was placed in the stretching device and stretched 15-20 mm (50-60% stretching) and left for 30 mins. After which the stretched PDMS was released which results in fragmented nanosheets of plasmonic thinfilms which provides high signal enhancement.

FIG. 11A shows SEM images of PDMS stretched and plain at 100×, 150× and 300× magnification. Optical images of PDMS membrane before and after silver plasmonic nanoparticle deposition are shown in FIG. 11B. It is observable that the stretched PDMS has stretch marks in the nanometer range compared to plain PDMS which has no stretch marks but random imperfections. Characterizations and testing performed on the CF substrate described in above examples were also carried out for the PDMS substrate. The preparation of the CF substrate for biomarker sensing was also carried out for the PDMS substrate.

### Example 13: Immunoassay Procedure for CF Membrane SERS

PDMS SERS substrates were cleaned with ethanol and water. The substrates were immersed in modified protein solutions of varying concentrations for 30 mins, after which the resultant substrates were rinsed in PBS and blocked with 0.5 mg/ml thiolated BSA in PBS for 30 mins. Blocked substrates were rinsed with PBS to remove unbound BSA protein and immersed in 1 mg/ml 4-ATP tagged antibody solution for 30 mins. Substrates were rinsed in PBS and water before measurement by a Raman spectroscopy system.

### Example 14: Performance of PDMS Membrane

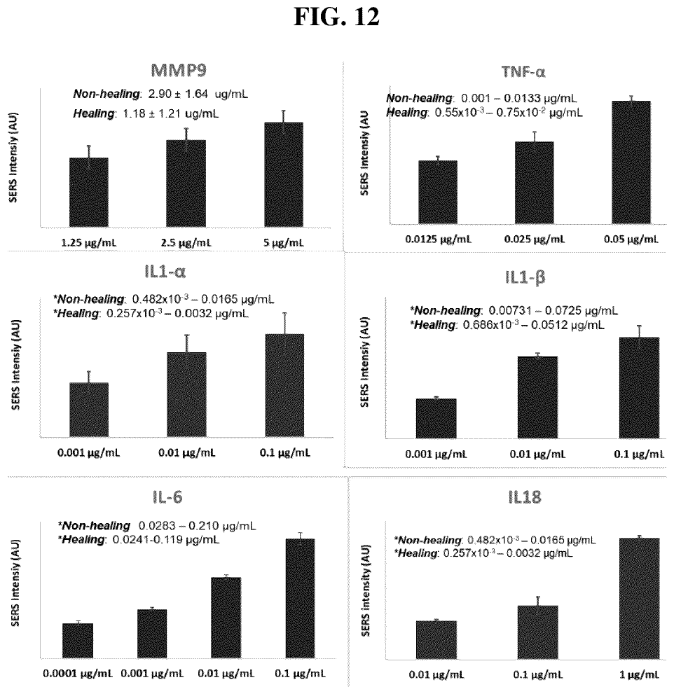

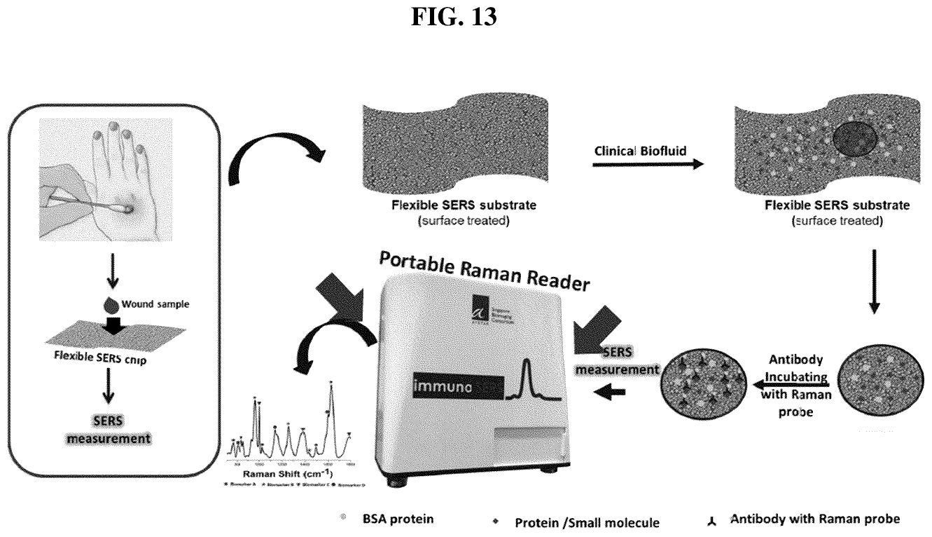

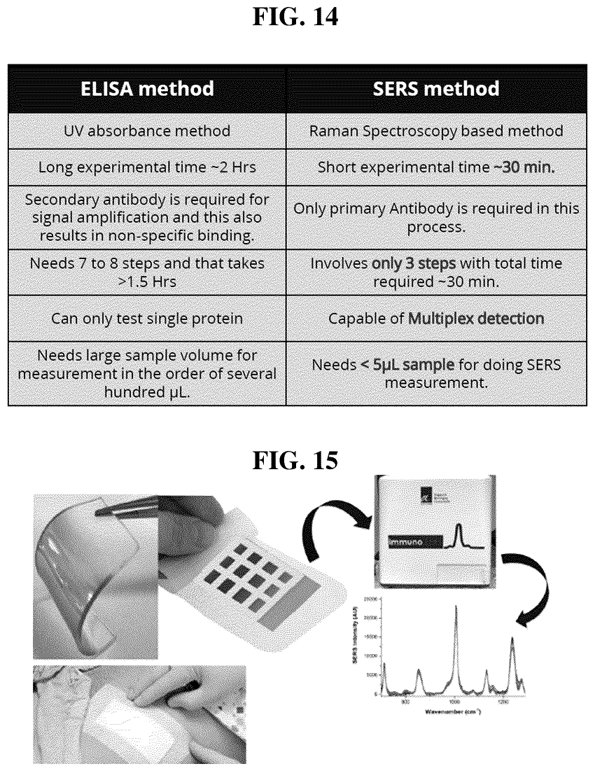

FIG. 12 shows the calibration plot obtained for different wound healing biomarkers using the present PDMS-based membrane flexible SERS substrate. Based on the experiments with both CF membrane and PDMS membrane substrates showed reliability in terms of linear correlation with respect to concentration of the biomarker.

### Example 15: Flexible Membrane-Based SERS for Multiplex Protein Detection

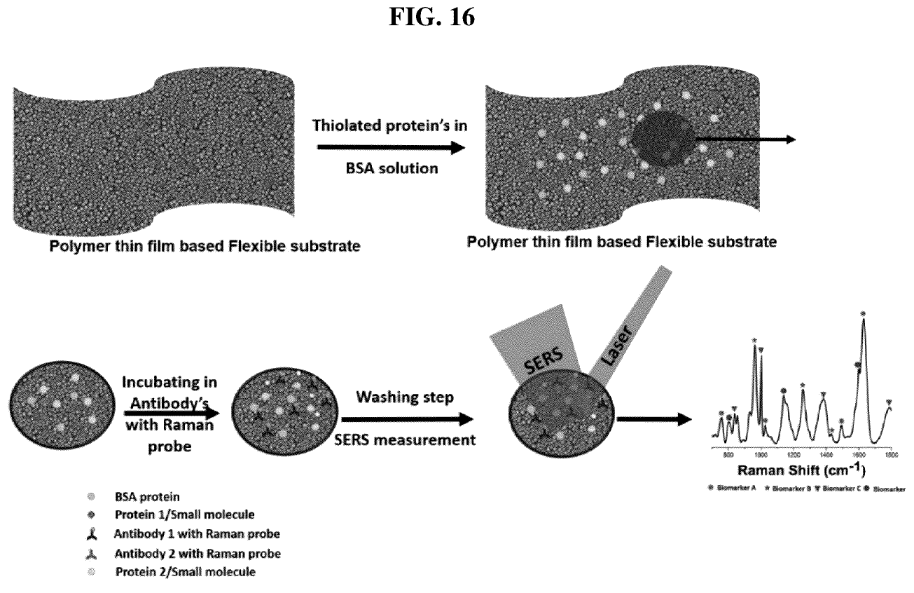

Following similar procedure as that of single protein detection, multiple protein mixture were treated with instant thiolating agent (Traut's reagent). To prevent non-specific binding of Raman reporter tagged antibodies, a BSA protein blocking step was performed. BSA protein is also treated with thiolating agent to improve uniform and stable blocking of active site of the SERS substrate. After this step, different antibodies tagged with various Raman reporter molecules were used to incubate with the protein and BSA immobilised flexible SERS substrate. Between each step, thorough washing step was done with PBS buffer to effectively remove unbound proteins and antibody. FIG. 16 shows the schematic illustration of the multiplex biosensing process. Finally, Raman microscope was used to detect and quantify SERS signal of different Raman reporter molecules from the resultant biosensor SERS membrane.

### Example 16: Summary, Commercial and Potential Applications

Wound management may have stretched the limits of health systems globally, challenging clinicians to evaluate the effectiveness of their treatments and deliver appropriate care to their patients. Visual inspection and manual measurement of wound size is subjective, often inaccurate and inconsistent. Growth factors, pro-inflammatory cytokines and proteases, such as tumor necrosis factor alpha (TNF-α) and interleukin one (IL1) alpha and beta, along with matrix metalloproteinasc (MMPs), play important roles in cutaneous wound healing. However, little is known about point-of-care monitoring of the changes in these growth factors, cytokines and enzymes during the healing process. In the present disclosure, the capability of surface-enhanced Raman spectroscopy (SERS) as a viable point-of-care platform to monitor changes of these surrogate indicators of healing status in chronic wounds.

Herein, the present method develops for a flexible, cost-effective, easy-to-fabricate, and scalable approach to fabricate plasmonic SERS substrates using, for example, cellulose fibers (CF) and PDMS. These substrates after activation may be incorporated into wound dressings to permit routine monitoring of wound status. This approach is superior to enzyme-based assays as the latter is time consuming, resource intensive and requires acquiring samples directly from wounds.

The present plasmonic membranes were evaluated and the reliable silver nano-island thickness that is sputtered onto the CF and PDMS substrate to enable it with highest SERS enhancement. This was achieved by depositing Ag nano-islands onto CF membranes at 20 mA for 60 seconds. Based on the surface activated CF-based SERS substrate, varying concentrations of MMP-9 (10-5000 ng/mL) and TNF-α (5-100 ng/ml) proteins to model wound exudates on the present plasmonic membranes were detected. The SERS biomarker detection method demonstrates linear responses within biologically relevant concentrations, ranging from 10 to 500 ng/ml for MMP-9, and in the case of TNF-α, non-linear response for the range of 5 to 100 ng/ml by achieving the highest detection point of sub-μg/mL for these biomarker proteins.

Diabetes mellitus (DM) is increasingly prevalent, and the monitoring of DM related wound healing poses challenges for clinicians to evaluate the effectiveness of their treatments. Growth factors and pro-inflammatory cytokines, such as tumour necrosis factor alpha (TNF-α), matrix metalloproteinase (MMP-9) and interleukin one (IL1) alpha and beta, may have a role in the chronic wound healing process that aids in removing impaired extracellular matrix components for new extracellular matrix molecules required for wound healing to progress. Herein, as a non-limiting example, the potential of using surface enhanced Raman spectroscopy (SERS) with polydimethylsiloxane (PDMS) and/or cellulose fiber (CF) based membrane as a viable point of care platform to monitor the changes of various cytokines, proteases and other small molecules as wound biomarkers were studied.

In the present disclosure, SERS as a sensitive and selective method to detect various wound biomarkers was demonstrated, given its ability to produce fingerprint spectra of its analyte rapidly at high resolution. A flexible and transparent PDMS and cellulose membrane-based SERS substrate decorated with randomly arranged silver nanostructures were fabricated as one of the examples for this study.

PDMS/cellulose membrane-based SERS substrates can be used for sample collection in wet condition of wound exudates and by employing a robust biosensing method to detect the biomarkers. Biomarkers such as cytokines and proteases were detected using the Raman active reporter tagged antibody by means of a modified ELISA protocol. 4-Aminothiophenol (4-ATP) was adopted as a non-limiting example of a Raman reporter molecule in this study. Detection of varying concentrations in the range of pg/ml to μg/ml of protein in phosphate buffered saline (PBS) that were spiked with BSA protein were achieved, wherein the PBS spiked with BSA protein was deposited onto the PDMS/cellulose membrane-based SERS substrate.

In the case of the small molecule, a label-free direct detection approach can be used without further sample processing followed by data processing to unmix individual biomarker data by means of machine learning. SERS using flexible substrate serves as an affordable and reliable platform with high specificity and sensitivity for point of care monitoring of trace biomarkers, to aid clinicians in assessing the progress of DM chronic wound healing.

The present plasmonic membranes serves as a sensing platform which achieved detection limits in the μM to sub-nM range and displayed high sensitivity and selectivity. The integration of plasmonic sensors in dressings and applying SERS to measure and report clinically relevant indicators, can result in a cheap, point-of-care device that provides non-invasive measures of cutaneous wound healing in real-time. Possible interest to wound care industry and to clinics attending to out-patients with diabetic, pressure and ulcer wounds.

While the present disclosure has been particularly shown and described with reference to specific embodiments, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

1. A biocompatible SERS-active polymer membrane configured to detect biomarkers in a sample, comprising:
a flexible and porous polymer membrane; and
SERS-active nanoparticles formed on the flexible and porous polymer membrane, wherein the flexible and porous polymer membrane comprises cellulose or an elastomeric polymer.

2. The biocompatible SERS-active polymer membrane of claim 1, wherein the elastomeric polymer comprises a silicon-based elastomer or the elastomeric polymer comprises polydimethylsiloxane.

3. The biocompatible SERS-active polymer membrane of claim 1, wherein the flexible and porous polymer membrane is functionalized with an anchoring agent to render an amine functional group or a mercapto functional group for binding to a SERS-active nanoparticle, wherein the anchoring agent comprises (3-aminopropyl) triethoxysilane, (3-mercaptopropyl) trimethoxysilane, or mercaptopropylsilatrane.

4. The biocompatible SERS-active polymer membrane of claim 1, wherein the SERS-active nanoparticle comprises silver, gold, or copper.

5. The biocompatible SERS-active polymer membrane of claim 1, wherein:
the SERS-active nanoparticles formed on the flexible and porous polymer membrane are spaced apart at 150 nm or less, and/or
the SERS-active nanoparticles formed on the flexible and porous polymer membrane have a thickness of up to 60 nm.

6. The biocompatible SERS-active polymer membrane of claim 1, wherein the biomarkers comprise a matrix metalloproteinase, tumor necrosis factor alpha, and/or an interleukin.

7. (canceled)

8. A method of forming the biocompatible SERS-active polymer membrane of claim 1, the method comprising:
providing a flexible and porous polymer membrane, wherein the flexible and porous polymer membrane comprises cellulose or an elastomeric polymer; and
depositing SERS-active nanoparticles on the flexible and porous polymer membrane.

9. The method of claim 8, wherein providing the flexible and porous polymer membrane comprises contacting the flexible and porous polymer membrane with an anchoring agent to have an amine functional group or a mercapto functional group functionalized thereon for binding to a SERS-active nanoparticle.

10. The method of claim 8, wherein the flexible and porous polymer membrane comprises an elastomeric polymer and the method further comprises stretching the flexible and porous polymer membrane up to 60% prior to depositing the SERS-active nanoparticles.

11. The method of claim 8, wherein depositing the SERS-active nanoparticles comprises:
placing the flexible and porous polymer membrane into a coater operable to deposit the SERS-active nanoparticles thereon; or
placing the flexible and porous polymer membrane onto an electron beam evaporator to deposit the SERS-active nanoparticles thereon; or
sputtering a precursor of the SERS-active nanoparticles onto the flexible and porous polymer membrane to have the SERS-active nanoparticles formed thereon.

12. The method of claim 11, wherein depositing the SERS-active nanoparticles is carried out at a deposition current of 10 mA to 20 mA.

13. The method of claim 8, wherein depositing the SERS-active nanoparticles is carried out for a duration of up to 60 s.

14. The method of claim 8, wherein depositing the SERS-active nanoparticles comprises depositing the SERS-active nanoparticles at a rate of up to 1 nm/s.

15. A method of determining a state of wound healing in a diabetic individual, the method comprising:
contacting a sample extracted from a wound of the diabetic individual with a thiolating agent to have one or more biomarkers suspected to be contained in the sample form one or more modified proteins;
incubating the biocompatible SERS-active polymer membrane of claim 1 with the one or more modified proteins to immobilise the one or more modified proteins on SERS-active nanoparticles of the biocompatible SERS-active polymer membrane;
providing one or more Raman probes attached with one or more antibodies, wherein each of the one or more antibodies binds specifically to one corresponding modified protein from the one or more modified proteins;
contacting the one or more Raman probes with the biocompatible SERS-active polymer membrane having the one or more modified proteins immobilised thereon; and
subjecting the biocompatible SERS-active polymer membrane to SERS spectroscopy to generate one or more SERS signals corresponding to the one or more biomarkers, respectively.

16. The method of claim 15, further comprising:
contacting the biocompatible SERS-active polymer membrane of claim 1 with a serum albumin to have the serum albumin immobilized on surfaces of the flexible and porous polymer membrane which are not occupied by the one or more modified proteins.

17. The method of claim 15, wherein the thiolating agent comprises a Traut's reagent.

18. The method of claim 15, wherein the one or more antibodies comprise an antibody of matrix metalloproteinase, an antibody of tumor necrosis factor alpha, and/or an antibody of interleukin.
